(19) United States
(12) Patent Application Publication
(10) Pub. No.: US 2009/0118573 A1
(43) Pub. Date: May 7, 2009

(54) Vacuum suction cup

(76) Inventor: Min-Hao Tsao, Taoyuan City (TW)

Correspondence Address: HDLS Patent & Trademark Services, P.O. BOX 220746, CHANTILLY, VA, 20153-0746, US

(21) Appl. No.: 11/934,882

(22) Filed: Nov. 5, 2007

(51) Int. Cl. A61F 5/00; A61H 19/00; A61H 7/00

(52) U.S. Cl. 600/38; 601/137; 601/14

(57) ABSTRACT

A vacuum suction cup made of silicate gel instead of conventional hard plastic material for boosting breasts and enlarging penis includes multiple massage grains arranged as desired on inner surface of the cup. The cup entirely indents and attaches to human skin to deliver massaging and kneading functions when the vacuum suction is turned on for providing effective stimulation to points where acupuncture is applied in human body.

### BACKGROUND OF THE INVENTION

1. Field of the Invention

The present invention is related to a vacuum suction cup, and more particularly, to a suction cup connected to a vacuum that covers up a specific position of a human body to provide vacuum suction and stimulate points where acupuncture to be applied in human body.

2. Description of the Prior Art

Practically all women would do anything to develop and maintain perfect curves. However some women due to congenital deficiency develop flat chests. Medical reports indicated proper suction of breasts could stimulate mammary glands thus to boost breasts. Therefore, vacuum suction devices have been introduced into the market. The vacuum suction device includes an air pump, piping and cup. The cup covers upon the breast and the air pump operates by manually or driven by power to apply proper suction on the breast in the hope to boost the breast.

The vacuum suction cup of the prior art is made of hard plastic material to provide suction in conjunction with the air pump. However the breast boosting result is very limited.

### SUMMARY OF THE INVENTION

The primary purpose of the present invention is to provide a vacuum suction cup made of soft material, e.g., silicate gel or similar material with proper resiliency. Multiple massage grains are disposed on an inner surface of the cup as desired. Accordingly, when the vacuum suction cup operates, the resilient cup vacuumed is deformed and entirely indented to attach to human skin to provide contact similar to direct massage and kneading effects in stimulating mammary glands and points where acupuncture is usually applied in the human body. The present invention is more effective for the purposes of breast boosting and penis enlargement than the prior art that works only by providing non-contact vacuum suction.

The shape of each of those massage grains however is not limited and may be made in nipple or rib shape and those massage grains may be arranged in a way or direction as applicable.

The inner side of the cup may be designed with a non-flushed structure, i.e., showing regular or irregular indentions and protrusions to achieve the similar massage effects.

### BRIEF DESCRIPTION OF THE DRAWINGS

FIG. 1 is an exploded view showing an operating status of a first preferred embodiment of the present invention.

FIG. 2 is a perspective view showing the first preferred embodiment of the present invention as assembled.

FIG. 3 is a schematic view showing that the first preferred embodiment is applied on a breast.

FIG. 4 is a schematic view showing that the first preferred embodiment is executing vacuuming when applied on the breast.

FIG. 5 is a schematic view showing a second preferred embodiment of the present invention is applied on a penis.

FIG. 6 is a schematic view showing an operating status with a cylinder of the second preferred embodiment of the present invention when applied on the penis.

FIG. 7 is a schematic view showing another preferred embodiment yet of multiple massage ribs disposed to the present invention.

FIG. 8 is a schematic view showing that the present invention is applied to tighten vagina muscle.

FIG. 9 is a schematic view showing that the present invention is applied in collecting breast milk for storage.

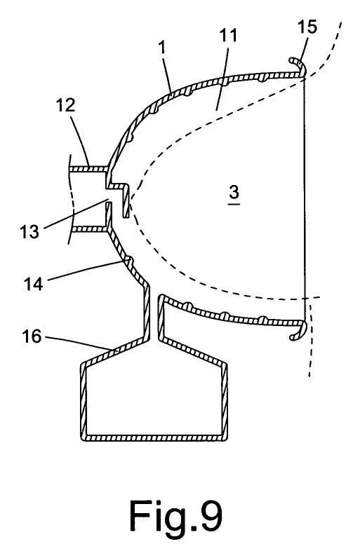

### DETAILED DESCRIPTION OF THE PREFERRED EMBODIMENTS

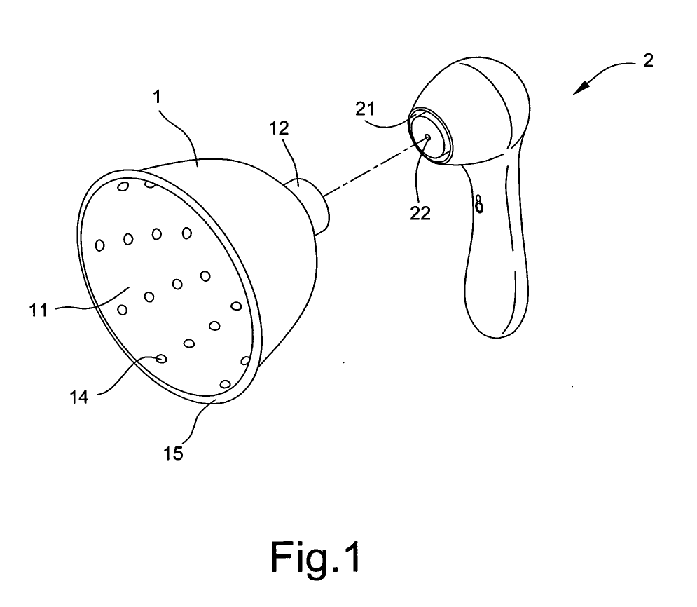

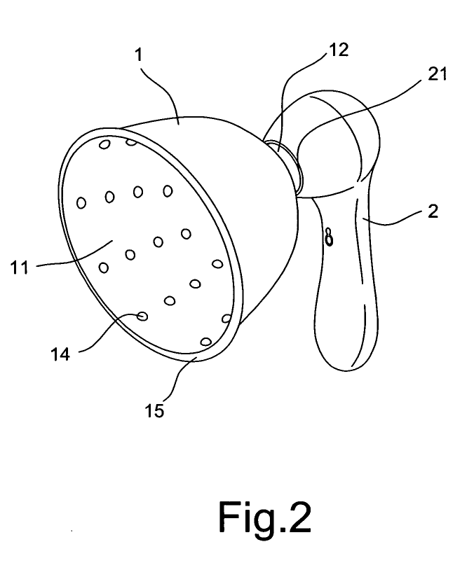

Referring to a preferred embodiment of the present invention as illustrated in FIGS. 1 and 2, a vacuum suction cup structure includes a cup 1 that can be connected to a vacuum suction device 2.

The cup 1 is made of soft material, e.g., silicate gel with a proper resiliency or similar material. When the cup 1 is applied in boosting a breast 3 and is made a hollow body in an approximately hemispherical shape containing a space 11. One end of the cup 1 is disposed with a connection portion 12 containing multiple through holes 13. On inner surface of the cup 1, i.e., peripheral to the space 11 is provided with multiple massage grains 14 each made in a protrusion or spherical shape in the preferred embodiment. The cup is provided with a flange 15 to contact the skin surrounding a breast, and the flange 15 is preferred to be made of a material slightly harder than that of the cup 1 so to prevent deformation in admitting air ingression to destroy vacuum status.

The vacuum suction device 2 functions as a pump and a connection seat 21 is disposed to the vacuum suction device 2 at where appropriately, and the connection seat 21 is provided with an air pump hole 22 in shape and construction not as characteristics to be claimed by the present invention.

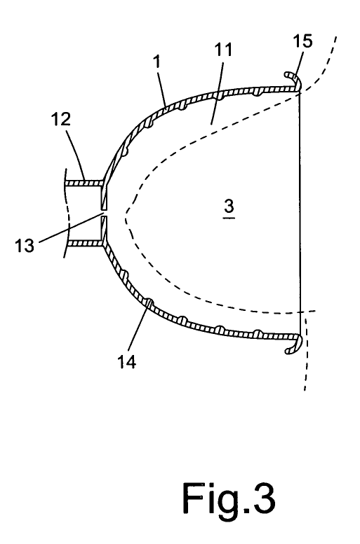

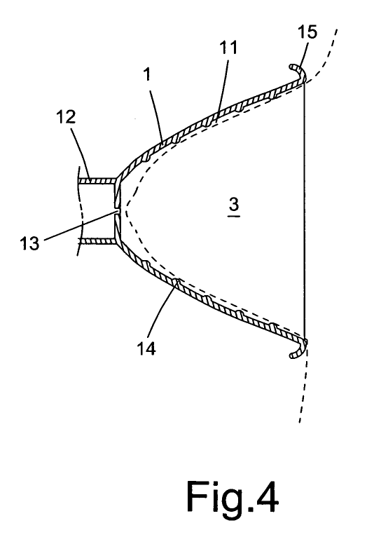

The cup 1, though made of a material with proper resiliency, maintains a given shape as illustrated in FIG. 1 under normal status. Now referring to FIG. 3 when the cup 1 is applied to a breast 3 for suction, the cup 1 covers upon the breast 3 and the cup 1 is connected to the vacuum suction device 2. Before vacuuming, a gap exists between the cup 1 and the vacuum suction device 2. As the vacuum suction device 2 is turned on, air inside the cup 1 is pumped out of the cup 1, and the cup 1 indents and deforms to tightly attach to the breast 3; meanwhile, those massage grains 14 provided on the inner side of the cup 1 directly contact the breast 3 to forthwith stimulate the mammary glands or points at where acupuncture is applied in human body as illustrated in FIG. 4. Furthermore, the vacuum status of the vacuum suction device 4 is intermittent, not continuous, that is, it cycles reciprocally vacuuming, pressure releasing, and draining air; therefore, the cup 1 also undergoes cycles of deformed indention and restoration to produce kneading and massage on the breast 3, a function that can never be achieved by the prior art.

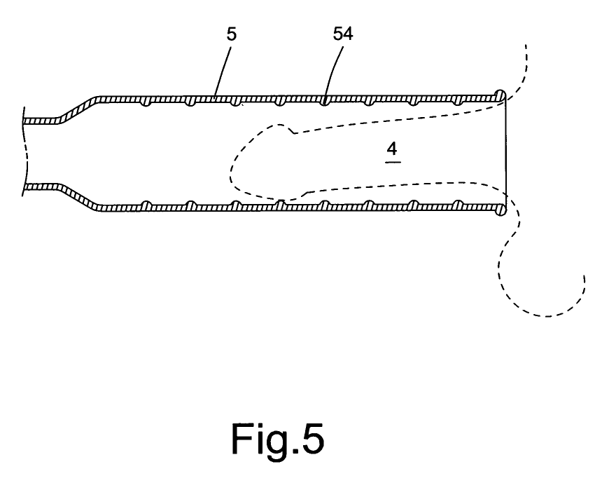

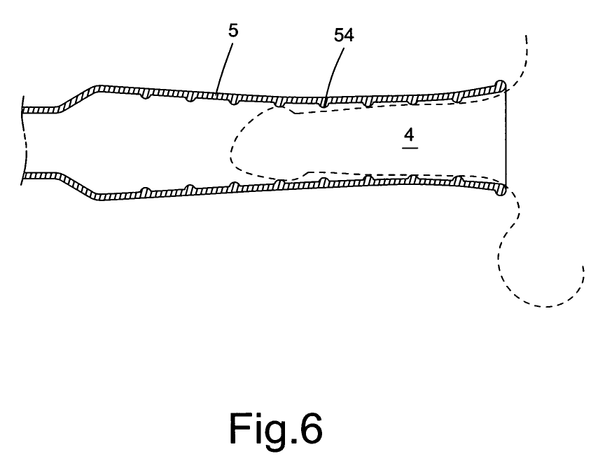

As illustrated in FIG. 5, the cup 1 is made into a cylindrical shape for the present invention to be applied for a penis 4. As illustrated in FIG. 5, a hollow cylinder 5 is made of the same resilient material and contains multiple massage grains 54. Upon vacuuming, the cylinder 5 properly indents as illustrated in FIG. 6 for those massage grains to directly stimulate points where acupuncture is usually applied on the penis 4 for health care and even enlargement purpose.

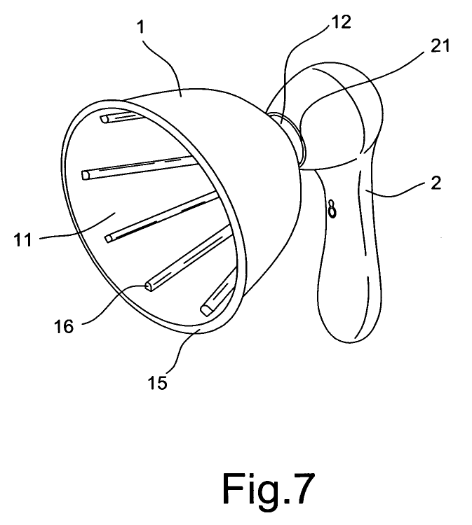

Now referring to FIG. 7, alternatively, each of those massage grains 16 is made into radius rib shape to realize the same massage results.

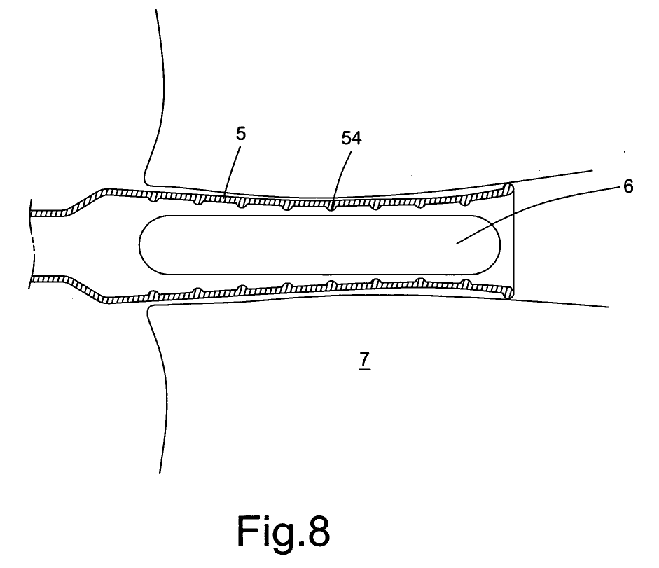

As illustrated in FIG. 8, the preferred embodiment according to FIG. 5 is modified for the vacuum suction cylinder 5 to be applied to a vagina. When a woman suffers slack vagina or incontinence, she can place the cylinder 5 in conjunction with a stick to take advantage of the repeated cycles of contraction and release applied on the vagina when the cylinder 5 is vacuumed to improve symptoms of slack vagina and incontinence.

Furthermore, as illustrated in FIG. 5, the cup 1 of the present invention may be disposed with another cup 16 to collect breast milk for storage. Accordingly, the present invention is capable of providing sufficient massage of the mammary glands to increase secretion of breast milk to facilitate nursing a baby.

I claim:

1. A vacuum suction cup made of resilient material that allows deformation, which comprise a hollow space, and multiple massage grains provided on an inner surface thereof; one end thereof being disposed with a connection portion and the connection portion being provided with a through hole.

2. A vacuum suction cup as claimed in claim 1, wherein the cup is adapted to a vacuum suction device.

3. A vacuum suction cup as claimed in claim 1, wherein the vacuum suction device is provided at where appropriately a connection seat, and the connection seat is provided with an air pump hole.

4. A vacuum suction cup as claimed in claim 1, wherein the cup is made in an approximately hollow hemispheric shape.

5. A vacuum suction cup as claimed in claim 1, wherein the cup is made in a cylindrical shape.

6. A vacuum suction cup as claimed in claim 1, wherein a flange of the cup is made of a material slightly harder than that of the cup for the cup to become a compound comprised of two types of materials in different resiliency.

7. A vacuum suction cup as claimed in claim 1, wherein the cup is made of silicate gel.

8. A vacuum suction cup as claimed in claim 1, wherein each of those massage grains is made in a form of protrusion, hemispheric, or rib, or any combination of them.

9. A vacuum suction cup as claimed in claim 1, wherein the inner surface of the cup is designed in a non-flushed structure indicating regular or irregular indentions and protrusion.

10. A vacuum suction cup as claimed in claim 1, wherein another cup to collect breast milk is disposed on the bottom of the cup.
